SEQUENCE LISTING (9 sequences)

SEQ ID NO: 1 - DNA, 29 bp, Artificial Sequence
  Oligonucleotide primer
cgactcactatagggcgaacccttactcc

SEQ ID NO: 2 - DNA, 29 bp, Artificial Sequence
  Oligonucleotide primer
ggagtaagggttcgccctatagtgagtcg

SEQ ID NO: 3 - DNA, 28 bp, Artificial Sequence
  Oligonucleotide primer
gagtaagggttcgccctatagtgagtcg

SEQ ID NO: 4 - DNA, 36 bp, Artificial Sequence
  Cloning site
ctcgaggaatgccgaccatggaaggcattcaagctt

SEQ ID NO: 5 - DNA, 39 bp, Artificial Sequence
  Oligonucleotide primer
ttacacgtcttgcggccgcgtgtaggctggagctgcttc

SEQ ID NO: 6 - DNA, 37 bp, Artificial Sequence
  Oligonucleotide primer
tgggaattaggcggccgccatatgaatatcctcctta

SEQ ID NO: 7 - DNA, 75 bp, Artificial Sequence
  Vector cloning region
gctcttcacctcgagcgattacagagaatgccgaccatggaaggcattcagactcatacaagcttcgagaagagc

SEQ ID NO: 8 - DNA, 25 bp, Artificial Sequence
  Oligonucleotide with 3′ phosphate
cctcgagcgattacagagaatgcct

SEQ ID NO: 9 - DNA, 26 bp, Artificial Sequence
  Oligonucleotide with 3′ phosphate
cgaagcttgtatgagtctgaatgcct

(19) United States
(12) Patent Application Publication
(10) Pub. No.: US 2011/0306099 A1
(43) Pub. Date: Dec. 15, 2011

(54) METHOD OF CLONING DNA

(76) Inventors: Matthew Beasley, Victoria (AU); Ben Kiefel, Victoria (AU)

(21) Appl. No.: 13/127,447

(22) PCT Filed: Oct. 30, 2009
(86) PCT No.: PCT/AU2009/001427

§ 371 Date: Aug. 29, 2011

(30) Foreign Application Priority Data
Nov. 3, 2008 (AU) 2008905669
Jul. 17, 2009 (AU) 2009903350

(51) Int. Cl. C12N 15/66; C12N 9/12

(52) U.S. Cl. 435/91.41; 435/194

(57) ABSTRACT

The present invention relates to a method for cloning double-stranded DNA (ds DNA) molecules. In particular, the present invention relates to a method for cloning ds DNA molecules using terminal transferase to tail at least one 3′ termini of the ds DNA molecules with nucleotides and ligating the tailed ds DNA molecules with a vector. Also provided are kits and compositions that can be used for cloning ds DNA molecules.

### FIELD OF THE INVENTION

The present invention relates to the field of DNA cloning, in particular, cloning of double stranded DNA molecules, for example, such as those generated by the polymerase chain reaction (PCR).

### BACKGROUND OF THE INVENTION

Since the polymerase chain reaction (PCR) was first described in the mid-1980s, it has become a ubiquitous and indispensible technique for molecular biologists, allowing the sensitive amplification, detection and cloning of DNA from their samples.

The thermostable polymerase most commonly used for PCR, Taq DNA polymerase from Thermus aquaticus, was found by Clark (1988) to add a single, non-templated, base to the 3′ end of a DNA duplex. For Taq polymerase this base is most commonly deoxyadenosine (dA), although the other three bases are added with a frequency dependent on the identity of the 3′ end nucleotide (Hu, 1993).

The addition of a non-templated single-base overhang occurs for many polymerases (Clark, 1988) and seems to be correlated with the absence of a 3-5′ proofreading activity. Therefore, while the presence of the single-base overhang can facilitate DNA cloning, it does so only for the products of polymerases that are intrinsically error prone. Commercially available proofreading polymerases (Pfu, Vent, Kod, Pfx, etc) all generate products with blunt ends.

Methods for direct cloning of PCR products are therefore dependent on the type of polymerase used for amplification, and therefore the type of end produced; either a single-base overhang or blunt.

Blunt cloning methods rely on high concentrations of ligase (T4 DNA ligase) and, generally, a negative selection system to eliminate the high proportion of recircularised plasmids that would otherwise result in a high background. Direct cloning of PCR products by blunt cloning is recognised as inefficient and slow.

Cloning of PCR products with a single-base overhang utilise vectors with a complementary overhang. A number of methods and commercial kits rely on this property for the direct cloning of PCR products. Complementary single-base overhangs found to enable direct cloning of PCR products from non-proofreading polymerases include; dT (U.S. Pat. No. 5,827,657; U.S. Pat. No. 5,487,993), dG (US 20080166773), uracil (U.S. Pat. No. 5,856,144) or dideoxythymidine (Holton and Graham, 1991).

One common feature of the blunt and single-base overhang protocols is a long ligation time—from 1 hour to overnight. These ligation times are relatively long compared to DNA cloning using restriction endonuclease sites which produce 4 base overhangs, allowing efficient ligation in as little as 5 minutes (cohesive end cloning). However, this requires that the PCR primer sequences be designed with extra terminal bases containing the restriction site (up to 12 bases for efficient cleavage) and also a more substantial time investment for cleavage with the restriction enzyme and post-digestion cleanup (1 to 2 hours).

Although the ligase activity of vaccinia Topoisomerase I (Topo I) (U.S. Pat. No. 5,766,891) provides rapid cloning of PCR products with either blunt or single-base overhangs, the requirement that the Topo I enzyme be covalently bound to vector ends means that this method of cloning is relatively costly.

There remains a need for methods to rapidly clone both blunt and base-overhang PCR products with high efficiency, desirably with similar efficiency and low rate of background achieved using cohesive end cloning.

### SUMMARY OF THE INVENTION

The present inventors have demonstrated that the 3′ termini of dsDNA molecules can be tailed with one or more nucleotides by terminal transferase, and the tailed dsDNA molecule can be subsequently cloned into a vector with complementary 3′ overhangs. In addition, the present inventors have found that tailing and ligation reactions can be performed in the same buffer either in sequential reactions or concurrently in the same reaction mixture, thus allowing for the rapid and efficient cloning of a dsDNA molecule. Components for tailing and cloning the dsDNA molecule can conveniently be provided in a kit.

Accordingly, the present invention provides a kit for cloning a dsDNA, the kit comprising nucleotides, terminal transferase and DNA ligase.

In a preferred embodiment, the nucleotides are ribonucleotides.

In one embodiment, the ribonucleotides are rGTP or rUTP.

In one particular embodiment, the ribonucleotides are rGTP.

In another embodiment, the kit comprises a mixture of nucleotides, for example a mixture of ribonucleotides and deoxyribonucleotides. For example, the kit may comprise rGTP, rATP and dATP.

In another embodiment, the kit further comprises a vector comprising at least one 3′ overhang.

In a preferred embodiment, the end of the at least one 3′ overhang of the vector comprises a chain-terminating base analogue or a 3′ phosphate. The chain-terminating base analogue may be for example a dideoxynucleotide or an acyclonucleotide.

In one embodiment, the chain-terminating base is ddTTP.

In one embodiment, the vector comprises at least one 3′ overhang of one to four bases.

In one particular embodiment, the vector comprises at least one 3′ overhang of three bases.

In another embodiment, the kit further comprises a buffer.

Although any suitable ligase known to the person skilled in the art may be used to ligate the dsDNA molecule to the vector, in one embodiment the DNA ligase is T4 DNA ligase.

The vector in the kit of the invention may be any vector which is able to replicate in a host cell and is suitable for cloning the dsDNA. In one embodiment, the vector is a plasmid.

In one embodiment, the kit further comprises competent host cells capable of replicating the plasmid. For example, the host cells may be bacteria such as E. coli.

In yet another embodiment, the kit comprises reagents for PCR.

The present invention further provides a composition comprising terminal transferase and DNA ligase.

In one embodiment, the composition further comprises a buffer.

In one particular embodiment, the DNA ligase in the composition is T4 DNA ligase.

The present invention further provides use of the composition of the invention for cloning a dsDNA molecule.

The present invention further provides a method of cloning a dsDNA molecule, the method comprising:

i) producing a dsDNA molecule comprising 3′ overhangs by contacting a dsDNA molecule with terminal transferase in the presence of ribonucleotides, and

ii) ligating the dsDNA molecule comprising 3′ overhangs to a vector comprising at least one complementary 3′ overhang.

In one embodiment, the dsDNA molecule comprises a single base 3′ overhang prior to contacting the dsDNA molecule with terminal transferase in the presence of ribonucleotides.

In another embodiment, the dsDNA molecule comprises blunt-ends prior to contacting the dsDNA molecule with terminal transferase in the presence of ribonucleotides.

In one embodiment, the dsDNA molecule comprising 3′ overhangs is ligated to the vector comprising at least one complementary 3′ overhang by ligating the dsDNA molecule to the vector with DNA ligase.

In one embodiment of the method of the present invention, one to four ribonucleotides are added to the 3′ ends of the dsDNA molecule and the vector comprises at least one complementary 3′ overhang.

In one particular embodiment, two ribonucleotides are added to the 3′ ends of the dsDNA molecule.

In one embodiment, contacting the dsDNA molecule with terminal transferase in the presence of ribonucleotides and ligating the dsDNA molecule to the vector are performed in the same buffer.

In another embodiment, contacting the dsDNA molecule with terminal transferase in the presence of ribonucleotides and ligating the dsDNA molecule to the vector are performed sequentially.

In a preferred embodiment, contacting the dsDNA molecule with terminal transferase in the presence of ribonucleotides and ligating the dsDNA molecule to the vector are performed concurrently in the same reaction mixture, wherein the at least one 3′ overhang of the vector is non-reactive with terminal transferase.

In one embodiment, contacting the dsDNA molecule with terminal transferase in the presence of ribonucleotides and ligating the dsDNA molecule to the vector are performed at about 2° C. to about 40° C., preferably at about 20° C. to about 40° C.

The present invention further provides a method of cloning a dsDNA molecule, the method comprising:

i) producing a dsDNA molecule comprising 3′ overhangs by contacting a dsDNA molecule with terminal transferase in the presence of nucleotides, and

ii) ligating the dsDNA molecule comprising 3′ overhangs to a vector comprising at least one complementary 3′ overhang,

wherein contacting the dsDNA molecule with terminal transferase in the presence of nucleotides and ligating the dsDNA molecule to the vector are performed concurrently in the same reaction mixture, and wherein the at least one 3′ overhang of the vector is non-reactive with terminal transferase.

In one embodiment, the nucleotides are deoxyribonucleotides.

In a preferred embodiment, the nucleotides are ribonucleotides.

In one specific embodiment, the ribonucleotides are rGTP.

In another embodiment, the nucleotides are a mixture of ribonucleotides and deoxyribonucleotides. For example, the nucleotides may be dATP, rGTP and rUTP.

In embodiments of the invention where the vector comprises at least one 3′ overhang which is non-reactive with terminal transferase, the end of the at least one 3′ overhang of the vector may comprise a chain-terminating base analogue or a 3′ phosphate. For example, in one embodiment, the chain-terminating base analogue may be a dideoxynucleotide or an acyclonucleotide.

In an embodiment, the chain-terminating base is ddTTP.

The dsDNA which is suitable for cloning using the method of the present invention may be any dsDNA molecule and may be of synthetic or genomic origin. For example, the dsDNA may be a PCR amplification product or a sheared genomic DNA fragment that has been suitably treated for cloning.

In a preferred embodiment, the dsDNA molecule is a PCR amplification product.

In one embodiment, the dsDNA molecule is a PCR amplification product which was PCR amplified with a non-proof reading DNA polymerase.

In one particular embodiment, the non-proof reading polymerase is Taq polymerase.

Any suitable vector may be used to clone the tailed dsDNA. In one embodiment, the vector is a plasmid.

In another embodiment, the method of the invention further comprises transforming a host cell with the vector.

In a preferred embodiment, the host cell is E. coli.

In the method of the invention, the dsDNA molecule is contacted with terminal transferase for about 5 minutes to overnight, preferably for about 1 hour, or more preferably for about 20 minutes.

The present invention further provides a method of cloning a dsDNA molecule, the method comprising:

i) producing a dsDNA molecule comprising 3′ overhangs by contacting a dsDNA molecule with terminal transferase in the presence of ribonucleotides,

ii) ligating the dsDNA molecule comprising 3′ overhangs to a vector comprising at least one complementary 3′ overhang, and

iii) transforming the dsDNA molecule ligated to the vector into a host cell.

The present invention further provides a method of cloning a dsDNA molecule, the method comprising:

i) producing a dsDNA molecule comprising 3′ overhangs by contacting a dsDNA molecule with terminal transferase in the presence of nucleotides,

ii) ligating the dsDNA molecule comprising 3′ overhangs to a vector comprising at least one complementary 3′ overhang, and

iii) transforming the dsDNA molecule ligated to the vector into a host cell,

wherein contacting the dsDNA molecule with terminal transferase in the presence of nucleotides and ligating the dsDNA molecule to the vector are performed concurrently in the same reaction mixture, and wherein the at least one 3′ overhang of the vector is non-reactive with terminal transferase.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE FIGURES

FIG. 1. Ribo-nucleotide tailing efficiency. Lane 1. 230*/236 duplex, dNTPs; Lane 2. duplex, rNTPs; Lane 3. duplex, rATP; Lane 4. duplex, rCTP; Lane 5. duplex, rGTP; Lane 6. duplex, rUTP; Lane 7. duplex, no TdT/rNTPs.

FIG. 2. rGTP and dCTP concentration dependence. Lane 1. 230* ; Lane 2. 230*+rGTP/TdT; Lane 3. 230*/236 duplex, just melted, rGTP/TdT; Lane 4. 230*/236 duplex, rGTP/TdT 0.1 mM; Lane 5. 230*/236 duplex, rGTP/TdT 0.5 mM; Lane 6. 230*/236 duplex, rGTP/TdT 1.0 mM; Lane 7. 230*/236 duplex, dCTP/TdT 0.1 mM; Lane 8. 230*/236 duplex, dCTP/TdT 0.5 mM; Lane 9. 230*/236 duplex, dCTP/TdT 1.0 mM; Lane 10. 230*, dCTP/TdT 1 mM.

FIG. 3. rGTP, dCTP and mixed ratio labelling. Lane 1. 230*; Lane 2. 230*, 1 mM rGTP/TdT; Lane 3. 230*/236 duplex; 1 mM rGTP/TdT; Lane 4. 230*/236 duplex, not quenched 1 mM rGTP/TdT; Lane 5. 230*/236 duplex, 1 mM dCTP/TdT; Lane 6. 230*, 1 mM dCTP/TdT; Lane 7. 230*/236 duplex, not quenched; Lane 8. not loaded; Lane 9. 230*/236 duplex, 10 μM dCTP, 0.5 mM GTP; Lane 10. 230*/236 duplex 50 μM dCTP, 0.5 mM GTP; Lane 11. 230*/236 duplex 100 μM dCTP, 0.5 mM GTP. Lane 12. 230*/236 duplex 200 μM dCTP, 0.5 mM GTP.

FIG. 4. Time and temperature dependence of rGTP tailing. Lane 1. 230*/236 duplex; Lane 2. 230*/236 duplex, quenched; Lane 3. 230*/236 duplex, 1 min at 37° C., 0.5 mM rGTP; Lane 4. 230*/236 duplex, 2 min at 37° C., 0.5 mM rGTP; Lane 5. 230*/236 duplex, 3 min at 37° C., 0.5 mM rGTP; Lane 6. 230*/236 duplex, 4 min at 37° C., 0.5 mM rGTP; Lane 7. 230*/236 duplex, 5 min at 37° C., 0.5 mM rGTP; Lane 8. 230*/236 duplex, 10 min at 37° C., 0.5 mM rGTP; Lane 9. 230*/236 duplex, 30 min at 37° C., 0.5 mM rGTP; Lane 10. 230*/236 duplex, rGTP pre-labelled control; Lane 11. 230*/236 duplex, denatured, cooled 2 min, then quenched; Lane 12. 230*/236 duplex, 5 min at 25° C., 0.5 mM rGTP; Lane 13. 230*/236 duplex, 10 min at 25° C., 0.5 mM rGTP; Lane 14. 230*/236 duplex, 20 min, 0.5 mM rGTP; Lane 15. 230*/236 duplex, 30 min, 0.5 mM rGTP.

FIG. 5. Blunt vs overhang rGTP/TdT duplex labelling at 25° C. Lane 1.blunt duplex (230*/235); Lane 2. overhang duplex (230*/236); Lane 3. blunt duplex, quenched; Lane 4. overhang duplex, quenched; Lane 5. blunt duplex+rGTP/TdT, 3 min at 25° C.; Lane 6. blunt duplex, 5 min at 25° C.; Lane 7. blunt duplex, 10 min at 25° C.; Lane 8. blunt duplex, 15 min at 25° C.; Lane 9. blunt duplex, 20 min at 25° C. Lane 10. blunt duplex, 25 min at 25° C.; Lane 11. blunt duplex, 30 min at 25° C.; Lane 12. overhang duplex, 20 mins at 25° C.

FIG. 6. Activity of T4 DNA ligase in buffers. Lane 1. 1 kb ladder; Lanes 2-9, ligation reactions using EcoRI-cut pBS KSII; Lane 2. no ligase; Lane 3. ligase buffer (New England Biolabs); Lane 4. NEB buffer 2+1 mM rATP; Lane 5. NEB buffer 4/CoCl₂+1 mM rATP; Lane 6. NEB buffer 4/CoCl₂+1 mM rGTP; Lane 7. NEB buffer 4/CoCl₂+1 mM rGTP, 1 mM rATP; Lane 8. NEB buffer 4/CoCl₂+1 mM rGTP, 0.5 mM rATP. Lane 9. NEB buffer 4/CoCl₂+1 mM rGTP, 0.1 mM rATP.

FIG. 7. ATP vs GTP ribotailing efficiencies. Lane 1. 230*/236 duplex; Lane 2. 230*/236 duplex, quenched; Lane 3. 230*/236 duplex, 0.1 mM rATP; Lane 4. 230*/236 duplex, 0.5 mM rATP; Lane 5. 230*/236 duplex, 1.0 mM rATP; Lane 6. 230*/236 duplex, 0.1 mM rGTP; Lane 7. 230*/236 duplex, 0.5 mM rGTP; Lane 8. 230*/236 duplex, 1.0 mM rGTP.

FIG. 8. TdT timecourse for dA-overhang vs dC-overhang on duplex substrates. Lanes 3-10, 13. rGTP/TdT labelling of duplex substrate for various timepoints. Lane 1. dA-overhang duplex (230*/235 dA tailed); Lane 2. dA-overhang duplex quenched (t=0); Lane 3. dA-overhang duplex , t=0.5 min; Lane 4. dA-overhang duplex, t=1 min; Lane 5. dA-overhang duplex, t=2 min; Lane 6. dA-overhang duplex, t=5 min; Lane 7. dA-overhang duplex, t=10 min; Lane 8. dA-overhang duplex, t=20 min; Lane 9. dA-overhang duplex, t=30 min; Lane 10. dA-overhang duplex, t=60 min; Lane 11. dC-overhang (230*/236) duplex; Lane 12. dC-overhang, quenched; Lane 13. dC-overhang, t=60 mins; Lane 14. dA-overhang, rGTP pre-labelled control; Lane 15. dC-overhang, rGTP pre-labelled control.

FIG. 9. ddTTP and TdT concentration dependence for end-labelling. Lane 1. 230*/236 duplex; Lane 2. 230*/236 duplex, quenched; Lane 3. 230*/236 duplex, 0.05 mM ddTTP, 1/20× dilution TdT; Lane 4. 230*/236 duplex, 0.05 mM ddTTP, 1/10× dilution TdT; Lane 5. 230*/236 duplex, 0.05 mM ddTTP, ⅕× dilution TdT; Lane 6. 230*/236 duplex, 0.1 mM ddTTP, 1/20× dilution TdT; Lane 7. 230*/236 duplex, 0.1 mM ddTTP, 1/10× dilution TdT; Lane 8. 230*/236 duplex, 0.1 mM ddTTP, ⅕× dilution TdT; Lane 9. 230*/236 duplex, 0.5 mM ddTTP, 1/20× dilution TdT; Lane 10. 230*/236 duplex, 0.5 mM ddTTP, 1/10× dilution TdT; Lane 11. 230*/236 duplex, 0.5 mM ddTTP, ⅕× dilution TdT; Lane 12. 230*/236 duplex, 0.1 mM ddTTP, ⅕× TdT, NEB4, no CoCl₂; Lane 13. 230*/236 duplex, quenched.

FIG. 10. The number of colony forming units produced versus reaction time for TOPO TA, pGEM-T and TdT (Alchemy) cloning reactions.

FIG. 11. The number of colony forming units produced versus reaction time for TOPO-TA and TdT (Alchemy) cloning reactions.

### KEY TO THE SEQUENCE LISTING

SEQ ID NO:1—Oligonucleotide primer.
SEQ ID NO:2—Oligonucleotide primer.
SEQ ID NO:3—Oligonucleotide primer.
SEQ ID NO:4—Cloning site.
SEQ ID NO:5—Oligonucleotide primer.
SEQ ID NO:6—Oligonucleotide primer.
SEQ ID NO:7—Vector cloning region.
SEQ ID NO:8—Oligonucleotide with 3′ phosphate.
SEQ ID NO:9—Oligonucleotide with 3′ phosphate.

### DETAILED DESCRIPTION

### General Techniques and Selected Definitions

Unless specifically defined otherwise, all technical and scientific terms used herein shall be taken to have the same meaning as commonly understood by one of ordinary skill in the art (e.g., in cell culture, molecular biology, recombinant DNA technology, microbiology, and biochemistry).

Unless otherwise indicated, the molecular biology, microbiology, cell culture, and biochemical techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present) and are incorporated herein by reference.

### Terminal Transferase

As used herein “terminal transferase” refers to a non-templated DNA polymerase known as terminal deoxynucleotidyl transferase (EC 2.7.7.31; also known as TdT, DNTT). “Terminal transferase” includes reference to enzymes with a non-templated polymerase activity identical, or similar, to TdT, or its variants; such as, by way of non-limiting example, the modified TdT enzyme described in US 20040043396. The enzyme can be purchased commercially and is usually produced by expression of the bovine gene in E. coli. Examples of commercial sources of terminal transferase are Finnzymnes, MBI Fermentas, New England Biolabs (NEB), Promega, Panvera, Sigma Biochemicals, and Roche Molecular Biochemicals.

The present invention provides a method for cloning of double-stranded DNA (dsDNA), for example dsDNA produced by PCR. The broad-substrate activity of the terminal transferase non-templated polymerase enzyme is used to add a limited number of nucleotides to the 3′ ends of the dsDNA.

In a preferred embodiment, the present invention utilises terminal transferase to add ribonucleotides (rNTPs) to the 3′ end of a dsDNA sequence that is to be cloned (known as ‘ribo-tailing’). Addition of ribonucleotides (rNTPs) by terminal transferase to a DNA substrate is efficient, as NTPs have been found to be almost an acceptable substrate as dNTPs, however, chain extension is unable to progress beyond a few (<5) ribonucleotides. The ribo-tailed DNA sequence can be ligated to vector DNA that has a complementary 3′ overhang end.

Terminal transferase may be used at any suitable concentration for tailing a dsDNA molecule. By way of example, terminal transferase may be used at a concentration of approximately 1 Unit of enzyme per 5 μl of reaction volume, however, higher and lower concentrations of terminal transferase are contemplated. For example, to enhance the terminal transferase reaction rate, higher concentrations of terminal transferase would be expected to provide more rapid ribo-tailing. Similarly, as terminal transferase exists in vivo as different sized proteolytic forms of the active enzyme, a more active form may be provided, such as described in US 20040043396. In addition, lower concentrations of terminal transferase could be used in longer reaction times. While shown to be optimally active at 37° C. by completely ribo-tailing a 3′ overhang dsDNA in 10 minutes, at 25° C. terminal transferase was able to completely ribo-tail dsDNA in 20 minutes, thus demonstrating that terminal transferase may be used over a range of suitable incubation temperatures. The person skilled in the art will readily be able to determine suitable conditions under which the terminal transferase is active. For example, reaction volumes, concentrations, times and temperatures would be determined by routine methods.

The present inventors herein describe a set of reaction conditions that uniformly ribo-tail the dsDNA substrate with two nucleotide bases (on a dsDNA substrate that has a dA 3′ overhang). However, the reaction conditions could be readily modified to enable 3, or more, nucleotide bases to be added by terminal transferase. For example, different buffer compositions (for example, potassium cacodylate buffer), CoCl₂ or nucleotide concentrations, and/or terminal transferase activities or longer periods of incubation could produce overhangs that have 3 or more nucleotide bases that may then be cloned into vector DNA with a complementary 3′ overhang.

Although terminal transferase is not as efficient at ribo-tailing blunt dsDNA as compared to dsDNA with a 3′ overhang, a minor proportion of blunt-ended dsDNA is ribo-tailed by terminal transferase within a few minutes, and >50% of dsDNA is tailed within 30 minutes. As would be understood by the skilled addressee, even where only a proportion of the dsDNA is ribo-tailed, this would still allow for the dsDNA to be cloned using the method of the invention.

Double-Stranded DNA (dsDNA)

The term “dsDNA” as used herein refers to a double-stranded polynucleotide or oligonucleotide which may be of synthetic or genomic origin. The dsDNA sequences may either be blunt or have a 3′ overhang. By way of non-limiting example, the dsDNA may be the product of DNA amplification, such as amplification of DNA by PCR, or the dsDNA may be genomic DNA that has been sheared and then blunted by enzymatic treatment.

PCR is a reaction in which replicate copies are made of a target polynucleotide using a pair of primers and a catalyst of polymerization, such as a DNA polymerase, and typically a thermally-stable polymerase enzyme such as Taq polymerase. Methods for PCR are known in the art, and are taught, for example, in “PCR” (Ed. M. J. McPherson and S. G Moller (2000) BIOS Scientific Publishers Ltd, Oxford). The dsDNA molecule to be cloned by the method of the invention includes dsDNA amplified by methods other than PCR as are known to those skilled in the art. Such methods include isothermal amplification methods and transcription-based amplification systems.

According to the method of the invention, ribo-tailing of dsDNA is preferably performed on dsDNA that has a single 3′ base overhang, which is a common amplification product of PCR using non-proofreading enzymes, such as Taq DNA polymerase.

The single base added to the 3′ end of DNA duplexes produced by PCR using non-proofreading enzymes such as Taq is most commonly dA. Although the dA 3′ base overhang is an optimal substrate for ribo-tailing, dsDNA molecules with a 3′ base overhang consisting of a nucleotide other than dA may be cloned using the method of the invention. For example, a dC 3′ base overhang on a dsDNA molecule may still be efficiently ribo-tailed and would perform well for cloning DNA duplexes according to the method of the invention. As the 3′ base overhang added to DNA duplexes by non-proofreading polymerases is dependent on the terminal 3′ base identity (Hu, 1993) as well as the polymerase used (Clark, 1988; Hu, 1993) then the method of the invention could be easily adapted to clone PCR products using different 3′ base overhangs. For example, US 20080166773 describes a method of cloning of PCR products using the proportion of PCR products that possess a dG 3′ base overhang instead of a dA 3′ base overhang.

Where it is desirable that a dsDNA be amplified in the presence of a proof-reading DNA polymerase, for example Pfu, Vent, Kod, Pfx, or Pwo DNA polymerase, the resulting dsDNA molecule is blunt-ended. If desired, the dsDNA molecule can be tailed with a single nucleotide by treating the dsDNA molecule with a non-proofreading enzyme. By way of example, a blunt-ended dsDNA molecule could be dA tailed with Taq DNA polymerase to produce a dsDNA with a single base 3′ overhang.

While terminal transferase is able to ribo-tail a dsDNA which comprises any single base 3′ overhang, the present inventors have found that a dsDNA molecule with a dA 3′ overhang is most effectively ribo-tailed by terminal transferase. As Taq DNA polymerase adds a dA 3′ overhang, the PCR products amplified with Taq polymerase are effectively cloned by the method of the invention.

### Nucleotides

Terminal transferase may be used to add any nucleotide to a dsDNA molecule to produce a dsDNA molecule with a 3′ overhang. As used herein, “nucleotide” refers to a base-sugar-phosphate combination. Nucleotides are monomeric units of a nucleic acid sequence (DNA and RNA). While any nucleotide may be used in the method of the invention, preferably, the nucleotide is a ribonucleotide.

Reference to ribonucleotides refers to nucleotides where the purine or pyrimidine base is linked to a ribose sugar. Naturally occurring ribonucleotides (rNTPs) have the purine bases, adenine (A) and guanine (G), and the pyrimidine bases, cytosine (C) and uracil (U). However, for the method of the invention, the natural rNTPs may be substituted for nucleotide derivatives (e.g. inosine) or unnatural nucleotides that are also utilised by the terminal transferase activity in extending the 3′ hydroxyl base of a blunt or overhanging DNA duplex by a uniform and limited number of nucleotides.

Under identical limiting reaction conditions, approximately 50% of dsDNA molecules are ribo-tailed with ATP or CTP, whereas complete ribo-tailing occurs with GTP or UTP, making GTP and UTP preferred nucleotides for use in the method of the invention. While GTP is an optimal substrate for terminal transferase ribo-tailing of single-base overhang dsDNA substrates, the skilled person will understand that any nucleotide (ATP, CTP, GTP or UTP) may be used to satisfactorily ribo-tail a dsDNA in the cloning method of the present invention.

The concentration of nucleotides used in a ribo-tailing reaction with terminal transferase can be readily determined by the skilled person. By way of non-limiting example, optimal ribo-tailing may occur with the nucleotide at a concentration of 0.5 mM to 1 mM. However, higher or lower concentrations of nucleotides can be used and still achieve significant ribo-tailing of the dsDNA molecule. For example, the nucleotide may be used in the reaction at a concentration of 0.1 mM or less as could be determined by the person skilled in the art. Where a lower concentration of nucleotides is used in the reaction, it may be necessary for example to increase the incubation time of the terminal transferase reaction.

The present inventors have also shown that low levels (approximately 10 μM) of dNTPs in the ribo-tailing reaction mixture can be tolerated without adversely affecting the ribo-tailing reaction. Thus, where dNTPs are retained with the dsDNA sample from a PCR reaction, or have been used post-PCR to tail a blunt product with a 3′ single base overhang (for example, dA tailing with Taq polymerase), the dNTPs will not interfere with the tailing of the dsDNA molecule with a ribonucleotide.

Cloning dsDNA

The present inventors describe a method for tailing a dsDNA molecule with terminal transferase and cloning the tailed dsDNA molecule by ligating it with a vector having complementary 3′ overhangs. In view of the teachings of the present specification, the skilled person would be able to readily determine suitable reaction conditions in order to ligate the dsDNA molecule with the vector. For example, the enzyme used to ligate the dsDNA to the vector, the buffer components and co-factors used in the reaction could all be readily determined by the skilled addressee.

While the use of any suitable enzyme to ligate the vector to the dsDNA is contemplated, preferably the enzyme is a DNA ligase. As used herein, the term “DNA ligase” refers to an enzyme that is capable of joining a strand of DNA to a strand of DNA, or of joining a strand of DNA to a strand of RNA, with a covalent bond to make a continuous polynucleotide strand. Non-limiting examples of DNA ligases include T4 DNA ligase, T7 DNA ligase, E. coli DNA ligase, B. stearothermophilus DNA ligase, T4 RNA ligase and T. brucei RNA ligase.

As would be known to the skilled person, to enhance the rate of DNA ligation, DNA condensation agents such as hexamine cobalt chloride and macromolecular crowding agents such as polyethylene glycol (PEG) are also widely used as enhancers of the ligation rate.

The present inventors have shown for the first time that both terminal transferase and DNA ligase are active in the same buffer and under the same reaction conditions. Thus, the terminal transferase tailing reaction and ligation of the tailed dsDNA to the vector can not only be performed in separate reactions, but can be performed sequentially in the same buffer. By the “same buffer” it is meant that two buffer solutions have essentially the same composition, for example the same concentration of salts, co-factors etc.

Preferably the reactions can be performed concurrently in the same reaction mixture. As used herein “concurrently in the same reaction mixture” means combining all reagents necessary for both the terminal transferase tailing reaction and the ligation reaction in the same reaction volume such that both reactions occur within the same incubation.

The preferred embodiment of the invention of conducting terminal transferase tailing and DNA cloning reactions concurrently in the same volume is achieved by using a buffer recipe that is optimal for both terminal transferase enzyme and for the T4 DNA ligase enzyme. By way of non-limiting example, one buffer in which both terminal transferase and T4 DNA ligase are both active is a buffer comprising 20 mM Tris-acetate, 50 mM potassium acetate, 10 mM magnesium acetate, 0.25 mM CoCl₂, pH 7.9 at 25° C. Other suitable buffers can be readily determined by the person skilled in the art.

Although concurrent terminal transferase tailing and DNA cloning reactions work in the presence of dithiothreitol, the present inventors found that terminal transferase activity is greater in the absence of dithiothreitol. Thus, in a preferred embodiment, the buffer does not comprise dithiothreitol.

As the terminal transferase enzyme extends the 3′ base overhang on a dsDNA substrate using ribonucleotides, an overhang is created that is complementary to a 3′ overhang on suitably prepared vector DNA which is also in the reaction volume. T4 DNA ligase (or another DNA ligase activity) would then ligate the ribo-tailed dsDNA ends and the vector ends, to form a sequence capable of host cell transformation.

The present inventors have found that T4 DNA ligase is capable of utilising the GTP nucleotide as a cofactor for ligation. Thus the terminal transferase and ligation reactions can be performed concurrently in a buffer comprising GTP without ATP. However, as optimal reaction conditions for T4 DNA ligase utilise ATP as a cofactor, terminal transferase tailing and ligase reactions may preferably be performed concurrently in a reaction mixture comprising ATP.

ATP at 1 mM concentrations is a less efficient substrate for terminal transferase for ribo-tailing. However, at low concentrations (0.1 mM ATP) ATP still provides optimal ligation rates for T4 DNA ligase as well as not visibly producing ribo-tailed 3′ base overhang duplexes. Therefore, the added efficiency and convenience of conducting a simultaneous ribo-tailing and ligation/cloning reactions in a single buffer are achieved using this embodiment of the method of the invention where the buffer comprises GTP and also comprises ATP at low concentration.

By way of example, the present inventors applied the method of the invention to the cloning of a PCR product produced by Taq DNA polymerase. It was demonstrated that the method was both rapid (a 30 minute reaction), and efficient, with a low background (5% of clones without insert) and high cloning efficiency (˜3.7×10⁵ cfu/μg insert). The following procedure and conditions are illustrative and provide a non-limiting example of cloning a PCR product that has been generated using a DNA polymerase that adds 3′ dA overhangs using the method of the invention:
1) perform PCR of the target sequence using a non-proofreading DNA polymerase such as Taq DNA polymerase
2) (optional) allow a 5 to 10 minute polishing/tailing step at the end of the PCR at 72° C.
3) electrophorese the PCR product on an agarose gel to allow visualisation and then excise and purify the DNA using standard methods; alternatively, the PCR product may be cleaned directly from the PCR reaction by silica column binding. Elute the PCR product from the column with 30 μL TE buffer.
4) perform the concurrent ribo-tailing and vector ligation reaction at 25° C. for 30 minutes, with the volumes described in Table 1
5) denature the DNA ligase and TdT enzymes by incubation at 70° C. for 15 minutes
6) transform either 5 μL of the ligation into 100 μL chemically competent E. coli by standard methods, or 1 μL into 50 μL of electrocompetent E. coli by standard methods.
7) plate the transformation onto LB agar plates with the appropriate selective agent

The skilled person will understand that the reaction conditions, for example reaction volumes, concentrations and times may be widely varied while still successfully cloning the PCR product according to the method of the invention.

TABLE 1
Illustrative reaction volumes for ribo-tailing and cloning of PCR
products with 3′ overhangs by the method of the invention

| Volume | Stock reagent |
|---|---|
| 1.25 μL | 10 × NEB TdT reaction buffer |
| 5.0 μL | 2.5 mM CoCl₂ |
| 2.5 μL | 10 mM GTP |
| 1.0 μL | 2.5 mM ATP |
| 7.25 μL | mQ water |
| 5.0 μL | PCR product |
| 2.0 μL | vector DNA (ddT-tailed, BsmI-cut; 50 ng/μL) |
| 0.5 μL | TdT enzyme (NEB, Finnzymes, or Fermentas) |
| 0.5 μL | T4 DNA ligase (NEB) |

### Vectors and Host Cells

A “vector” may be any vector that is capable of transforming a host cell. Preferably, the vector is also capable of replicating within the host cell independently of the host's genome. Vectors can be either prokaryotic or eukaryotic, and include plasmids, viruses and cosmids as well as linear DNA elements, such as the linear phage N15 of E. coli, and/or extrachromosomal DNA that replicates independently of a host cell's genome.

Plasmid DNA may also be functionally separated before transformation, such that a functional sequence is assembled following ligation of the dsDNA to be cloned, either in vitro, or in vivo. For example, U.S. Pat. No. 7,109,178 describes a method of cloning using separate vector arms that are ligated to a DNA duplex using the vaccinia topoisomerase I enzyme. The functional vector is formed by a site-specific reaction between loxP sites at the ends of the vector arms by the Cre recombinase.

In one embodiment, the vector is an expression vector. By “expression vector” it is meant that the vector is capable of transforming a host cell and of effecting expression of a specified polynucleotide molecule in the cell.

Expression vectors may contain regulatory sequences such as transcription control sequences, translation control sequences, origins of replication, and other regulatory sequences that are compatible with the recombinant cell and that control the expression of polynucleotide molecules. In particular, recombinant vectors may include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in a suitable host cell. A variety of such transcription control sequences are known to those skilled in the art.

For concurrent ribo-tailing and vector ligation reactions, the ends of the vector DNA in the reaction should be resistant to modification by terminal transferase. This can be achieved, for example, through conducting a ribo-tailing reaction using the complementary nucleotide to the concurrent ribo-tailing/ligation nucleotide (for example using CTP for vector ribo-tailing and using GTP for DNA insert ribo-tailing) prior to the cloning reaction. Alternatively, a complementary non-reactive overhang could be achieved by the addition of a chain terminating base analogue, for example, the terminal addition of dideoxynucleotides or other suitable chain-terminating nucleotide analogue, to an overhang to form the complementary overhang. As another alternative, an oligonucleotide with a complementary 3′ ribonucleotide extension may be ligated to suitably prepared vector DNA to provide complementary ends for PCR cloning.

The dideoxynucleotide unnatural base analogues (ddNTPs) lack a 3′ hydroxyl which prevents further strand extension by a polymerase. For the ribo-tailing reaction for cloning a PCR product with a dA 3′ base overhang, the sequence ‘-dArGrG’ would be the 3′ overhang produced under the reaction conditions described in the Examples section herein (although different reaction conditions, e.g. buffer salts, enzyme and nucleotide concentrations, may yield different ribo-tails). Therefore, a vector with a ‘-dCdCddr 3’ overhang would be of complementary sequence and ligatable to the ribo-tailed PCR product comprising ‘-dArGrG’. Other chain-terminating base analogues, such as, by way of non-limiting example, include the acylonucleotides (acyNTPs) sold by New England Biolabs.

By way of non-limiting example, a suitable vector for use in the method of the present invention may be prepared by ligating a cassette containing two oppositely orientated BsmI sites, which produce a 3′ overhang of ‘CN’, into a plasmid vector. The sites are sequence designed to produce ‘CC’ 3′ overhang ends upon BsmI cleavage. Terminal transferase is used to add the ddT residue to the 3′-dCdC vector overhang similarly to the method described by Holton and Graham (1991) for the ddTTP 3′ tailing of vector DNA for cloning of dA-overhang PCR products. The ddTTP tailing reaction using terminal transferase may be performed, for example, in terminal transferase buffer for 10 minutes at 37° C. using only 0.1 mM ddTTP and 3 units of terminal transferase enzyme.

Another example of a vector suitable for use in the method of the invention is a vector having one or more 3′ overhangs that have a 3′ phosphate. The 3′ phosphate protects the vector from further terminal transferase modification during the terminal transferase reaction.

Suitable host cells for use in the cloning method of the invention include any host cell that can be transformed with a polynucleotide vector as are known to the person skilled in the art. Suitable host cells include animal, plant, bacterial, fungal (including yeast), parasite, and arthropod cells. Examples of suitable bacterial host cells include Escherichia coli and Bacillus subtilis. Other suitable host cells include Acinetobacter baylyi or the yeasts Saccharomyces cerevisiae or Schizosaccharomyces pombe. In a preferred embodiment, the host cell is E. coli.

### Kits

The necessary components for performing the cloning method of the invention may conveniently be provided in the form of a kit. In the kit based on the method of the invention, reaction components (for example, enzymes, salts, nucleotides and/or vector DNA) could be included in a buffer mixture, which could be supplied as a concentrate.

As will be understood to a person skilled in the art, the various components in the kit may be supplied in individual containers or aliquots, or the solution components may be combined in different combinations and at different concentrations to achieve optimal performance of the cloning method of the invention. It is within the knowledge of the skilled addressee to determine which components of the kit may be combined such that the components are maintained in a stable form prior to use.

The kits of the invention will typically at a minimum comprise terminal transferase and a DNA ligase. Preferably the kit further comprises nucleotides and a buffer concentrate in which, when diluted to a working concentration, both the terminal transferase and DNA ligase are both at least partially active. By “at least partially active” when used in relation to terminal transferase implies that at least a proportion of the dsDNA molecules in the reaction are tailed with nucleotides, and when used in relation to DNA ligase implies that at least a proportion of the tailed dsDNA molecules in the reaction are ligated to a vector. In a preferred embodiment, the kit further comprises a vector with 3′ overhangs suitable for cloning a tailed dsDNA molecule.

As would be understood by the skilled person, the buffer supplied with a kit will typically be supplied as a concentrate, for example a 10× concentrate, however the buffer may also be supplied at a lower concentration or at or near working concentration. By way of non-limiting example, a buffer which is suitable for use with the kit of the invention may comprise CoCl₂, rNTP, potassium acetate, Tris-acetate and magnesium acetate. Other additional components may be included with the kit, or other components supplied by the end user, if required.

### EXAMPLES

### Example 1

### Ribo-Tailing of a Fluorescent Duplex Substrate

To assay for terminal transferase (TdT) ribo-tailing of DNA duplexes one strand of a short (<30 base pairs) duplex was tagged with a fluorophore (fluorescein, FAM) that would be detectable using the SYBR green filter of the Fujifilm LAS-3000 chemidoc system.

The oligonucleotide pair 230* (5′ FAM-CGACTCACTATAGGGCGAACCCTTACTCC 3′ (SEQ ID NO:1)) and 235 (5′ GGAGTAAGGGTTCGCCCTATAGTGAGTCG 3′ (SEQ ID NO:2)), which form a 29 base pair duplex with a blunt 3′ end; and 230* and 236 (5′ GAGTAAGGGTTCGCCCTATAGTGAGTCG 3′ (SEQ ID NO:3)), which form a 28 base pair duplex with a single-base dC 3′ overhang were synthesised (Geneworks, Adelaide). An unlabelled oligonucleotide, 246, which was identical in base sequence to 230* was also synthesised and used in all assays in stoichiometric excess to prevent the fluorophore-labelled strand from reforming a duplex upon denaturation (‘quenched’ 230*).

The duplex was formed by adding equimolar amounts of the fluorescent labelled oligonucleotide 230* with either 235 or 236.
10 μL 10× NEB buffer 2
10 μL 230*; 50 μM in TE buffer
10 μL 235 or 236; 50 μM in TE buffer
70 μL mQ water

The mixture was then denatured at 70° C. for 2 minutes and annealed at 25° C. for 10 minutes. A ribo-tailing reaction was then assembled using NEB buffer 4, CoCl₂, NTP(s), the oligonucleotide duplex and TdT enzyme. New England Biolabs supply NEB buffer 4 as the reaction buffer for their TdT enzyme and it was used as the TdT reaction buffer for all experiments. 1× NEB buffer 4 is;
20 mM Tris-acetate
50 mM potassium acetate
10 mM Magnesium Acetate
1 mM Dithiothreitol
pH 7.9 at 25° C.

Different sources of TdT enzyme were purchased from New England Biolabs (M0315S), Finnzymes (F-203L) and Fermentas (EP0161) and, although having slightly different specific activities (15-20 U/μL), were nonetheless found to be essentially equivalent in NEB buffer 4 and used interchangeably.

To compare the different substrate properties of the four ribonucleotides, ATP, CTP, GTP and UTP, for ribo-tailing a duplex substrate containing a single 3′ base overhang, a reaction was assembled as follows;
2 μL 10× NEB buffer 4
2 μL 10× CoCl₂ (2.5 mM)
2 μL 10 mM rNTP/NTP
1 μL 5 μM oligonucleotide 230*/236 duplex (3′ dC base overhang)
12 μL mQ water
1 μL TdT enzyme

The reaction was conducted at 37° C. for 30 minutes. The labelled oligonucleotide (230*) was made single stranded by addition of an excess (3 μL of a 50 μM solution) of the quench oligonucleotide, 246, and 4 μL of 6× DNA loading dye and heated to 65° C. for 1 minute.

5 μL of the reaction was loaded onto a 20% polyacrylamide gel (PAGE) BioRad ‘minigel’, buffered with 1× TBE, and electrophoresed at a constant 160 V for 2 hours. The fluorescent oligonucleotide 230* was visualised in situ in the gel, without further treatment, using a Fujifilm LAS-3000 chemidoc system (SYBR green filter, 2 second exposure).

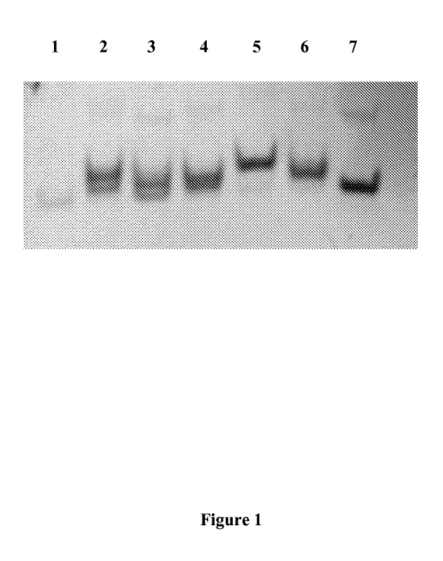

FIG. 1 demonstrates that the 3′ overhang is labelled by dNTPs (lane 1—most of the substrate did not enter the gel lane). rATP appeared a poor substrate, with only ˜50% of the oligonucleotide labelling (lane 3 rATP vs lane 7 unlabelled control). rCTP (lane 4) appeared to have 2 bands, also indicating incompletely labelled substrate. In contrast, both rGTP (lane 5) and rUTP (lane 6) appeared to label to completion with a single, shifted band. The notable shift for rGTP implies that it has had more than 1 nucleotide added to the 3′ overhang.

### Example 2

### Conditions of DNA Duplex Ribo-Tailing using rGTP

To further characterise the ribo-tailing of the overhang duplex with rGTP, the concentration dependence of the nucleotide vs labelling efficiency was examined. Furthermore, as TdT generally prefers single-stranded substrates to duplexes, the labelling efficiencies of each were compared.

Ribo-tailing reactions were assembled as described in Example 1, for a final concentration of 0.1 mM, 0.5 mM and 1.0 mM of either rGTP or dCTP. The oligonucleotide was either 230*/236 duplex or 230* alone. The labelling reaction was conducted at 37° C. for 30 minutes, before being quenched, and electrophoresed, as described for Example 1.

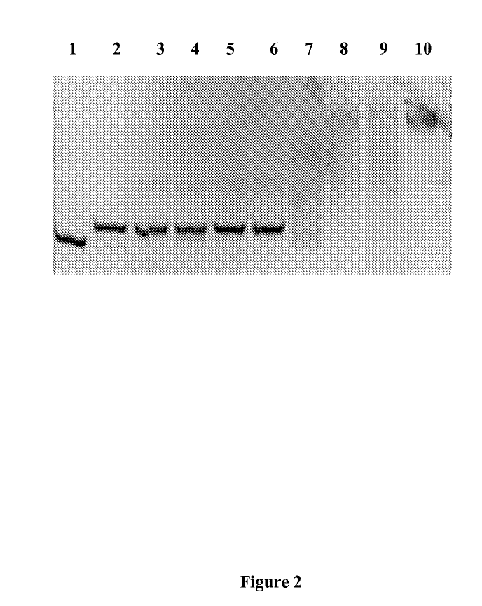

The results, shown in FIG. 2, demonstrate that both the single-stranded 230* oligonucleotide, as well as the 3′ overhang duplex, were efficiently and equally ribo-tailed using 1 mM rGTP. rGTP above 0.5 mM appeared to be in excess. However, using dCTP, the single-stranded substrate was more efficiently and consistently tailed than the 3′ overhang duplex (lane 9 vs lane 10).

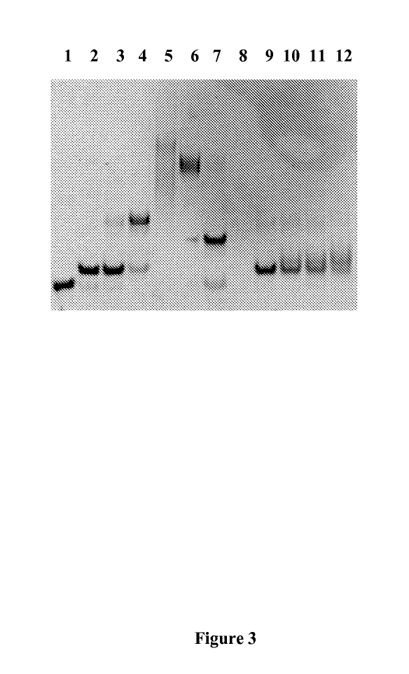

A ribo-tailing reaction using 1 mM rGTP was assembled as described above. FIG. 3 demonstrates that the 230* oligonucleotide was almost fully paired and double-stranded in the tailing reaction (lane 4 vs lane 7). In addition, the ribo-tailing reaction using 0.5 mM rGTP with levels of competing dCTP at 10 μM, 50 μM, 100 μM and 200 μM were conducted (lanes 9-12). This rGTP/dCTP co-labelling demonstrates that, at low dCTP concentrations (10 μM), TdT fully labels the duplex as a single discrete product (lane 9), presumably with ribo-tails of rGTP, which is at 50-fold higher levels than dCTP. At higher concentrations, a more variable range of products is observed, with presumably a mixture of rGTP and dCTP tails obtained. Only reaction conditions that produce an overhang tail of predictable sequence on DNA duplex substrates (e.g. TdT labelling using 0.5 mM rGTP, <10 μM dCTP) would be optimal for cloning as the vector tails need to be of a known, complementary sequence.

This result demonstrates that the method of the invention, using ribo-tailing to clone PCR duplexes, can tolerate low levels (˜10 μM) of dNTPs that may be retained from the PCR reaction, or used post-PCR to tail a blunt product with a 3′ single base overhang (e.g. dA tailing with Taq DNA polymerase).

1 mM rGTP was shown in FIG. 2 to be sufficient for full 3′ overhang duplex labelling using 1 μL TdT (10-15 units) in a 20 μL reaction volume, at 37° C. for 30 minutes. Therefore, to further define the reaction rates and conditions for full duplex ribo-tailing, the time and temperature dependence of TdT activity were examined.

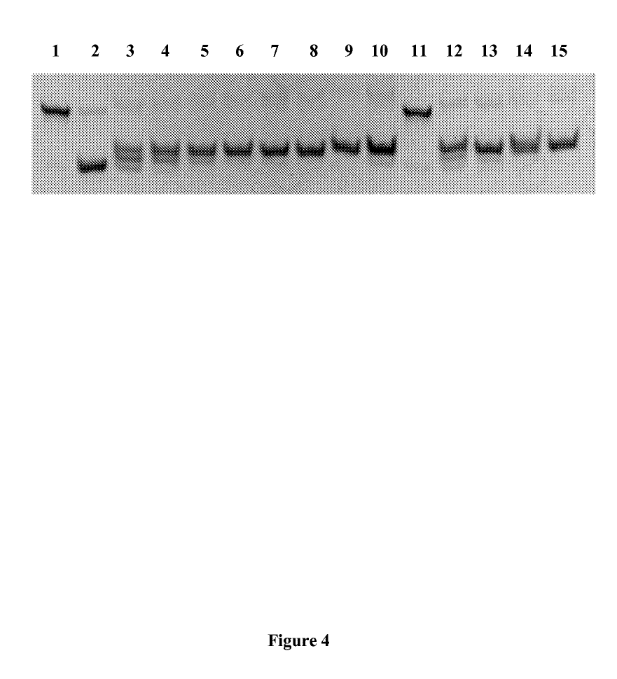

A timecourse experiment was conducted with reactions at 37° C. and 25° C. 5 μL samples were withdrawn at the timepoints (FIG. 4) and stopped by addition of 1 μL of 6× DNA loading dye and 0.5 μL quench oligonucleotide 246, and heated to 70° C. for 15 minutes. The ribo-tailing reaction was assembled as follows:
10 μL 10× NEB buffer 4
10 μL 2.5 mM CoCl₂
10 μL 5 μM duplex oligonucleotide, 3′ overhang (230*/236)
5 μL 10 mM rGTP
65 μL mQ water
2.5 μL TdT (Fermentas 20 U/μL)
60 μL was incubated at 37° C. 30 μL was incubated at 25° C.

FIG. 4 demonstrates that 1 unit of TdT enzyme per 5 μL sample was sufficient to enable complete ribo-tailing of the 3′ overhang duplex substrate in 10 minutes at 37° C., or 20 minutes at 25° C. Interestingly, at the shorter timepoints (1 minute at 37° C., lane 3; 5 minutes at 25° C., lane 12) there are clearly 2 discrete bands that, without being limited by theory, probably represent 1 and 2 guanosine base additions to the 3′ end of the labelled oligonucleotide. Therefore, it appears that, under these labelling conditions, a dinucleotide guanosine base ribo-tail is formed in a relatively short period (20 minutes at 25° C.).

### Example 3

### Ribo-Tailing of Blunt vs 3′ Overhang DNA Duplex Substrates

To determine whether the method of the invention could be used to clone blunt PCR products efficiently, a 29 base pair blunt duplex was formed from 230* and 235 oligonucleotides. The blunt DNA duplex was ribo-labelled in a reaction with 22 units of TdT (Fermentas) for various timepoints at 25° C.;
5 μL 10× NEB buffer 4
5 μL 2.5 mM CoCl₂
5 μL 5 μM oligonucleotide duplex (blunt or overhang)
2.5 μL 10 mM rGTP
33 μL mQ water
1.2 μL TdT (Fermentas)

5 μL aliquot samples were removed at timepoints at the reaction stopped by addition of 1 μL of 50 μM quench oligonucleotide (246) and 2 μL 6× DNA loading dye, and heated to 70° C. for 15 minutes.

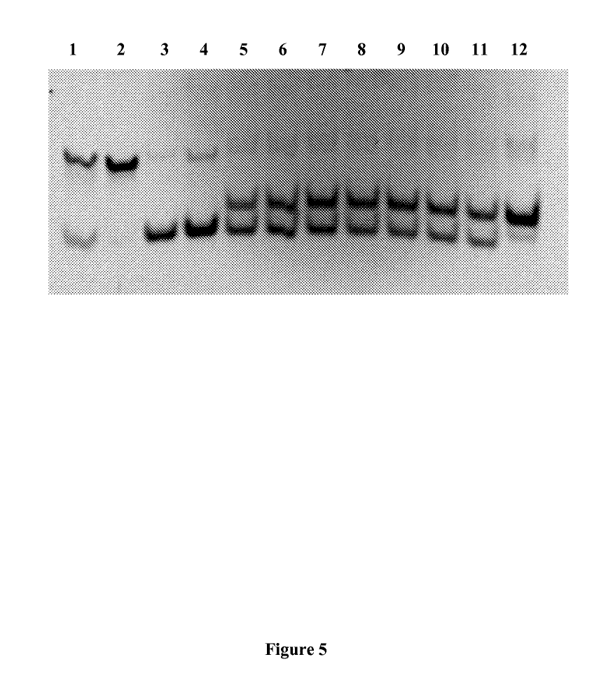

FIG. 5 demonstrates that the blunt duplex is resistant to ribo-tailing, with a minor proportion of the reaction, most probably any unpaired single-stranded labelled oligonucleotide, rapidly ribo-tailing in the first few minutes (lane 5), with a much slower reaction for the remainder of the duplex substrate. However, after 30 minutes, >50% of the duplex has been ribo-tailed, which would allow DNA cloning.

### Example 4

### T4 DNA Ligase Activity Under TdT Ribo-Tailing Conditions

For a preferred embodiment of the invention, concurrent ribo-tailing and DNA ligation in the same reaction volume, the predominant DNA ligase used for molecular cloning, T4 DNA ligase, should retain activity in the reaction conditions optimal for TdT. The activity of T4 DNA ligase in TdT reaction buffer (1× NEB buffer 4, 1 mM rGTP, 0.25 mM CoCl₂) was therefore tested.

A 3.0 kb plasmid, pBluescript KS II, was linearised with EcoRI, purified by binding to, and elution from, a silica column, and used as a substrate for T4 DNA ligase. Test ligation reactions were assembled as follows:
2 μL 10× buffer
(either NEB's T4 ligase buffer, NEB buffer 2, or NEB buffer 4)
2 μL linearised plasmid substrate
+/−2 μL 2.5 mM CoCl₂
+/−2 μL 10 mM rGTP
+/−X μL 10 mM rATP
0.5 μL T4 DNA ligase (NEB)
mQ water to a total 20 μL volume

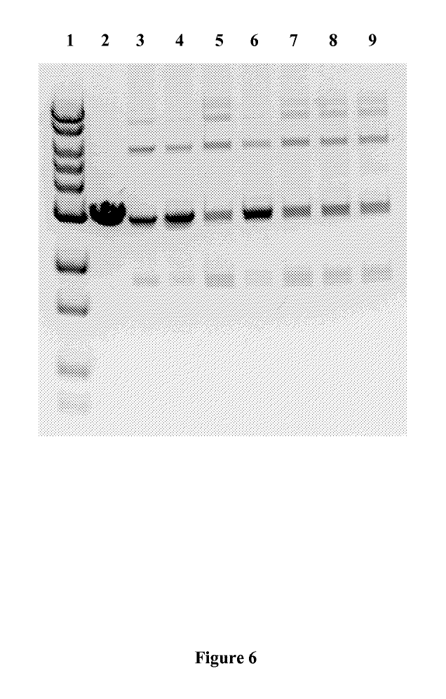

Ligations were incubated at 25° C. for 15 minutes then stopped by incubation at 65° C. for 20 minutes. FIG. 6 demonstrates that T4 DNA ligase is in fact optimally active in the TdT reaction buffer (lanes 6, and 7-9). Surprisingly, it is also capable of utilising the rGTP nucleotide as a cofactor for ligation (lane 6), although the preferred cofactor remains rATP (lane 5 vs lane 6). Ligation rates remain close to an optimal level even with 0.1 mM ATP (lane 9), vs the 1 mM levels normally supplemented in commercial ligase buffer preparations (lane 7).

As optimal reaction conditions for T4 DNA ligase are shown to be 1× NEB buffer 4, 1 mM GTP, 0.1 mM ATP, it was then necessary to determine whether the TdT enzyme would incorporate ATP at appreciable levels into the ribo-tail (although previous results had suggested that this would not be the case—see FIG. 1).

Ribo-tailing reactions using the 3′ overhang substrate 230*/236 were conducted with 0.1, 0.5 and 1 mM of ATP or GTP, using 4 units of TdT per 20 μL reaction, and incubated for 30 minutes at 25° C. Reactions were assembled and analysed as described in Example 1.

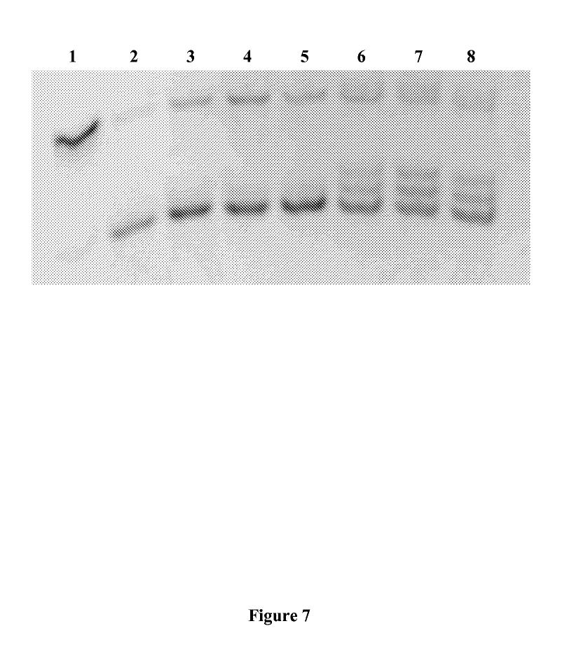

FIG. 7 demonstrates that ATP, unlike GTP, was a poor substrate for ribo-tailing at even 1 mM concentrations (TdT activity was limiting). Therefore, under the conditions determined, concurrent ribo-tailing and DNA ligation reactions would be expected to efficiently utilise GTP for the ribo-tailing reactions using TdT, and for the DNA cloning reactions to utilise ATP/GTP using T4 DNA ligase.

### Example 5

### Effect of Identity of 3′ Single Base Overhang on TdT Ribo-Tailing Efficiency

The non-templated base overhang added to the 3′ termini of PCR products by non-proofreading DNA polymerases is dependent on both the identity of the terminal base and the polymerase (Hu, 1993; Clark, 1988). To determine the difference in ribo-tailing efficiency between a 3′ dC and dA overhang using TdT polymerase, if any, the blunt substrate duplex (230*/235) was tailed with a dA base using Klenow exo⁻ (NEB, M0212L).
10 μL 10× NEB buffer 2
10 μL 230* oligonucleotide, 50 μM
10 μL 235 oligonucleotide, 50 μM
45 μL mQ water
5 μL 2 mM dATP
2 μL Klenow exo⁻ (5 U/μL, NEB)

dA tailing of the duplex substrate was performed at 37° C. for 1 hour. Tailing was stopped by heating the reaction to 70° C. for 20 minutes. The efficiency of the reaction was determined by comparing dA tailed 230* oligonucleotide to unlabelled on a 20% PAGE gel. Labelling was found to be 100% (not shown). Ribo-tailing using rGTP on both dC and dA 3′ overhang base substrates was performed using 4 units of TdT per timepoint.
2 μL NEB buffer 4
2 μL 2.5 mM CoCl₂
1 μL 5 μM 3′ overhang duplex oligonucleotides (dA or dC overhang)
1 μL 10 mM rGTP
12 μL mQ water
2 μL TdT (2 units/μL)

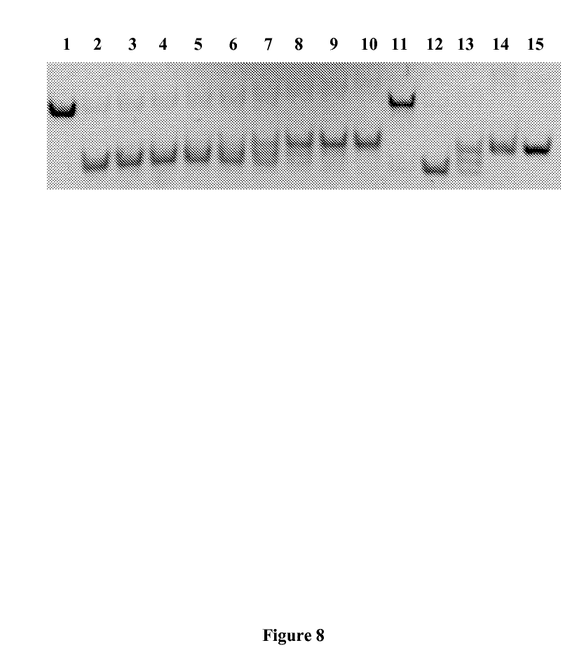

FIG. 8 demonstrates that TdT labels a dsDNA possessing a 3′ overhang deoxyadenosine (dA) base more efficiently than if a deoxycytosine (dC) base is present (lane 10 vs lane 13). As Taq DNA polymerase predominantly adds a dA 3′ overhang, then PCR products should be efficiently cloned by the method of the invention.

### Example 6

### Vector 3′ End Labelling with ddTTP and TdT

In a preferred embodiment of the invention, which is a combined TdT/T4 DNA ligase reaction enabling concurrent 3′ overhang tailing and DNA ligation, the vector end should be protected against modification by TdT enzyme otherwise it, itself, will be ribo-tailed during the reaction.

Holton and Graham (1991) describe labelling of 3′ ends of vector DNA by the non-extendable base analogue, ddTTP, a dideoxyribonucleotide. This was shown to enable cloning by single dA 3′ base overhangs (T/A cloning).

To enable the preferred embodiment of the invention, a cloning vector was designed with two oppositely orientated sites for the asymmetric restriction endonuclease, BsmI, which leaves a 3′ CN overhang (GAATGACNA). A two base CC overhang was designed as the ribo-labelling using GTP had shown two discrete bands in reactions with limiting TdT activity where the substrate was incompletely tailed (e.g. FIG. 8). The new cloning site was inserted into pBluescript KS II via XhoI and HindIII ends, and is shown below:

(SEQ ID NO: 4)
5′ ctcgaggaatgcc\hat{ }gaccatggaagg\hat{ }cattcaagctt
3′ gagctccttac\hat{ }ggctggtacctt\hat{ }ccgtaagttcgaa

The vector so formed, with the inverted BsmI sites for cloning by the method of the invention, was called pAthena.

To determine the conditions for ddTTP labelling using TdT, the test duplex substrate 230*/236 with a dC 3′ overhang was labelled with 0.05 mM, 0.1 mM and 0.5 mM ddTTP. The TdT activity was also varied for each ddTTP concentration, with 1.5, 3 and 6 units of TdT (Finnzymes) used per reaction. Reactions were incubated at 37° C. for 30 minutes and stopped by adding DNA loading dye and 246 quench oligonucleotide and heated at 65° C. for 15 minutes before electrophoresis and analysis.
2 μL 10× NEB buffer 4
2 μL CoCl₂
2 μL 5 μM oligonucleotide duplex (230*/236)
X μL ddTTP stock (0.5 mM or 5 mM)
2 μL TdT stock ( 1/20×, 1/10×, ⅕× dilution of Finnzymes TdT)
mQ water to 20 μL

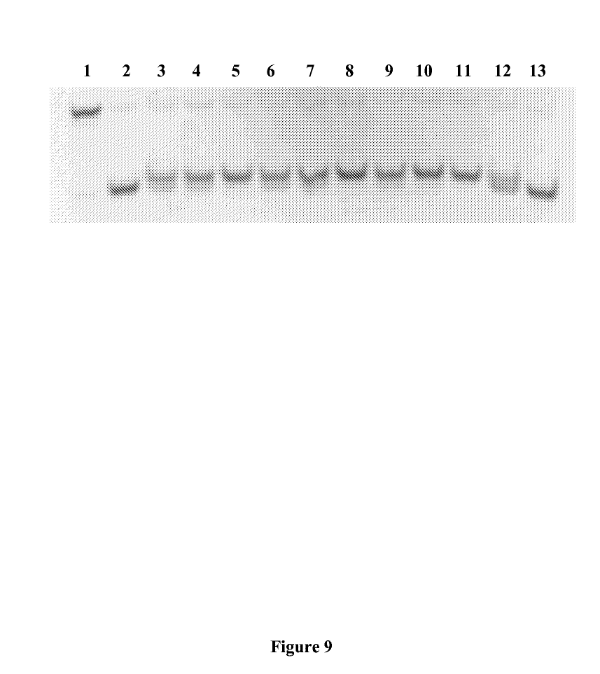

FIG. 9 demonstrates that ddTTP is efficiently added to the duplex substrate at even low concentrations of ddTTP and low levels of TdT activity (0.1 mM ddTTP, 6 units of TdT; lane 7) for ˜10 pmol of oligo ends.

### Example 7

### Cloning of Taq Polymerase PCR Products using Concurrent Ribo-Tailing and Ligation

A 1 kb gene for chloramphenicol resistance was amplified by Taq DNA polymerase (GoTaq, Promega) using the host-restrictive vector pKD3 (Datsenko and Wanner, 2000) and oligonucleotide primers

(SEQ ID NO: 5)
F′ 5′ TTACACGTCTTGCGGCCGCGTGTAGGCTGGAGCTGCTTC
and
(SEQ ID NO: 6)
R′ 5′ TGGGAATTAGGCGGCCGCCATATGAATATCCTCCTTA.

10 μL 5× GoTaq buffer
5 μL 2 mM dNTPs
1 μL F′ primer, 50 μM
1 μL R′ primer, 50 μM
1 μL pKD3 template (˜10 ng)
31.5 μL mQ water
0.5 μL GoTaq DNA polymerase (Promega, 5 units/μL)
   The PCR cycle program was:
1) 94° C. 30 seconds
2) 94° C. 20 seconds
3) 50° C. 20 seconds
4) 72° C. 40 seconds; steps 2-4; ×25 cycles
5) 72° C. 5 minutes

The PCR product was electrophoresed on a 1% agarose gel, excised, bound and eluted from a silica column in 30 μL of 10 mM Tris, 1 mM EDTA, pH 8 buffer. The cloning vector containing the inverted BsmI sites (pAthena) was prepared by BsmI digestion and ddTTP tailing as follows;
4 μL 10× NEB buffer 4
10 μL pAthena vector DNA (˜1 μg)
25 μL mQ
1 μL BsmI (10 U/μL)

The vector was cut with BsmI for 2 hours at 65° C. BsmI was inactivated by heating at 80° C. for 20 minutes. The vector was ddTTP tailed by adding to the digest:
8 μL 2.5 mM CoCl₂
1 μL 10× NEB buffer 4
1 μL TdT enzyme (Finnzymes, 15 units)
1 μL 5 mM ddTTP (GE lifesciences)

The ddTTP tailing reaction was incubated for 1 hour at 37° C. The vector DNA was then bound/eluted from a silica column to remove enzyme and free ddTTP. The chloramphenicol PCR product was cloned into the ddTTP-tailed, BsmI-cut vector using both sequential and concurrent ribo-tailing and ligation reactions.

### Sequential Cloning

ribo-tailing using GTP
2 μL 10× NEB buffer 4
2 μL 2.5 mM CoCl₂
2 μL 10 mM GTP
4 μL ChlR PCR product (˜200 ng)
9.5 μL mQ water
1 μL TdT (Finnzymes, 15 units)

Ribo-tailing was performed for 30 minutes at 25° C. At this point, the ligation reaction reagents were added directly to the existing ribo-tailing reaction.

PCR Insert Cloning using T4 DNA Ligase
0.5 μL 10 mM ATP
0.5 μL T4 DNA ligase (NEB)
2 μL pAthena vector (˜120 ng; BsmI cut, ddTTP tailed)

The ligation reaction was performed for 25 minutes at 25° C. The enzymes were inactivated by heating at 65° C. for 20 minutes.

### Concurrent Ribo-Tailing and Vector Ligation

2.5 μL 10× NEB buffer 4
5.0 μL 2.5 mM CoCl₂
2.5 μL 10 mM GTP
4.0 μL ChlR PCR (˜200 ng)
7.5 μL mQ water
1.0 μL TdT (Finnzymes, 15 units)
0.5 μL 10 mM ATP
0.5 μL T4 DNA ligase (NEB)
2.0 μL pAthena vector (˜120 ng; BsmI cut, ddTTP tailed)

The concurrent ribo-tailing and ligation reaction was incubated at 25° C. for 30 minutes and inactivated by heating at 65° C. for 20 minutes. A control reaction was also performed with vector DNA and enzymes, but without insert (ChlR PCR product).

A further control was performed with a vector containing a 3′ ‘CCC’ overhang produced by inverted Bgll sites. It was similarly treated to pAthena by ddTTP tailing and gel purification. This control was intended to demonstrate the selectivity of the ribo-tailing reaction under the conditions performed, to produce a dinucleotide ‘GG’ tail, and not a trinucleotide ‘GGG’ tail.

The ligations were transformed into electrocompetent E. coli cells (transformation efficiency of 5×10⁸ cfu/n) without further cleanup using 1 μL of heat-inactivated ligation mixture per 50 μL of thawed cells. The mixture was electroporated in a 1 mm cuvette at 1.8 kV and 1 mL of SOC recovery media added. The culture was incubated with shaking at 37° C. for 30 minutes and 100 μL ( 1/10^{th} of recovery volume) plated onto LB+ampicillin (100 μg/mL) plates.

Table 2 demonstrates that the method of the invention, concurrent ribo-tailing and ligation, resulted in hundreds of clones containing the desired insert, with a very low background (272 clones vs 15 on control plates). 10 colonies were chosen at random for plasmid analysis and all 10 were found to have the desired insert.

As a further test, an aliquot of the concurrent ribo-tailing ligations were incubated at 25° C. for 60 minutes before thermal inactivation. Although there was an increase in efficiency (350 cfu for 1 hour vs 272 cfu for 30 minutes) the 30 minute incubation is demonstrably adequate for almost all purposes. The cloning efficiency for the 1 kb insert was found to be ˜3.5×10⁵ cfu/μg (1 hour concurrent reactions).

TABLE 2
Cloning of a 1 kb PCR product using the method of the invention

| Ligation | Conditions | Cfu (ampR) |
|---|---|---|
| 1 | pAthena, sequential (30 mins) | 89 |
| 2 | pAthena, concurrent (30 mins) | 272 |
| 3 | pAthena, concurrent (1 hour) | 350 |
| 4 | pAthena, no insert control | 15 |
| | concurrent (1 hour) | |
| 5 | ‘CCC’ plasmid, sequential | 12 |
| 6 | ‘CCC’ plasmid, concurrent (30 mins) | 5 |
| 7 | ‘CCC’ plasmid, no insert control, | 2 |
| | concurrent (1 hour) | |

### Example 8

### Optimisation of Reaction Conditions

### Buffer Composition

The present inventors found that although terminal transferase is active in buffer comprising dithiothreitol, the activity of the enzyme is greater in buffer that does not comprise dithiothreitol. In addition, testing of different concentrations of NEB terminal transferase reaction buffer indicated that 0.5× NEB buffer is optimal. Thus, a preferred buffer for use in the cloning methods described herein is:
10 mM Tris-acetate
25 mM potassium acetate
5 mM magnesium acetate
5 mM cobalt chloride
1 mM GTP
0.1 mM ATP
pH 7.9, 25° C.

### Enzyme Ratio

Different ratios of terminal transferase (TdT) to T4 DNA ligase were tested in cloning reactions to determine the optimal ratio of these enzymes. TdT (NEB, M0315) and T4 DNA ligase (NEB, M0202S) units for optimal cloning performance were titrated at ratios of 1:4 (TdT:T4 DNA ligase) to 4:1.

The reactions were performed as per standard conditions (20 minutes at 25° C., 5 minutes at 65° C.) in a 10 μL total reaction with 100 ng of vector (2.3 kb) and 120 ng of insert (800 by test fragment). 1 of 10 μL ligation was electroporated into Argentum cells, of a 10⁸ cfu/μg competency. Following the plating of electroporated cells, the number of colony forming units per test reaction were counted (see Table 3).

TABLE 3
Colony forming units (CFU) produced with different ratios of enzyme

| TdT (μL) | T4 DNA ligase (μL) | #CFU |
|---|---|---|
| 0.1 | 0.4 | 336 |
| 0.2 | 0.3 | 1,360 |
| 0.3 | 0.2 | 1,500 |
| 0.4 | 0.1 | 840 |

A ratio of approximately 1:1 for TdT:T4 DNA ligase was found to be optimal for cloning. Higher concentrations of both enzymes may further improve cloning efficiency.

### Heat Inactivation

The present inventors found that there was an improvement to the yield of colonies per μg plasmid DNA if the Klenow and/or TdT/ligase reactions were heat inactivated by treatment at 65° C. for 5 minutes.

### Example 9

### Comparison of Cloning Techniques

The cloning method of the present invention was compared with Topo (Invitrogen; TOPO TA) and dT (Promega; pGEM-T) cloning kits. TOPO TA and pGEM-T reactions were performed according to the manufacturer's instructions.

### Topo Vector Cloning

1 μL diluted salt solution
2.5 μL PCR insert (600 by ChlR; 20 ng/μL)
1.5 μL mQ
1 μL vector
5 minutes at R/T
place on ice
pGEM-T Vector Cloning
5 μL 2× ligation buffer
1 μL pGEM-T vector
3 μL PCR insert (600 bp; 20 ng/μL)
1 μL T4 DNA ligase
30 minutes at R/T, take 3 μL sample and heat kill (65° C., 5 mins)
60 minutes at R/T, take 3 μL sample and heat kill (65° C., 5 mins)

### TdT Vector Cloning

0.5 ρL 10× TdT buffer (NEB)
2 μL 2.5 mM CoCl2
1 μL 10 mM GTP, 1 mM ATP solution
1 μL pAthena vector (80 ng/μL)
5 μL PCR insert (600 bp; 20 ng/μL)
0.25 μL TdT (NEB)
0.25 μL T4 DNA ligase (NEB)
25° C. for various time points, heat killed at 65° C. for 5 mins

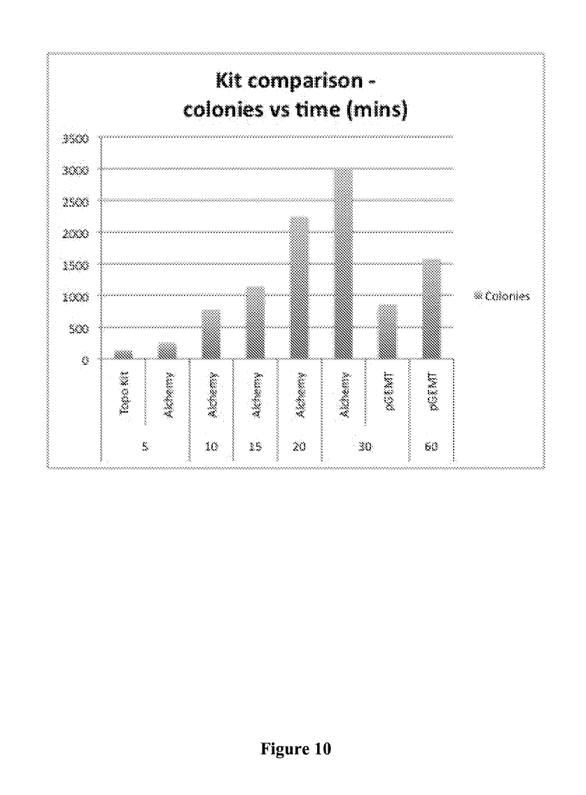

1 μL of each reaction was electroporated into Argentum cells (efficiency: 10⁹ cfu/μg). 1 mL of recovery media was added, and 100 μL of this volume was plated onto LB+ampicillin plates. Following overnight incubation at 37° C., the colonies on each plate were counted (where possible) and 10 colonies were screened for the PCR insert by colony PCR (Table 4 and FIG. 10).

TABLE 4
Comparison of cloning systems

| Cloning system | Cfu/plate | Inserts/10 colonies |
|---|---|---|
| Topo TA cloning | 138 | 10 |
| (rapid, low salt); | | |
| 5 mins | | |
| pGEM-T; 30 mins | 860 | 9 |
| pGEM-T; 60 mins | 1572 | 9 |
| TdT; 5 mins | 247 | 9 |
| TdT; 10 mins | 774 | 7 |
| TdT; 15 mins | 1140 | 8 |
| TdT; 20 mins | 2440 | 9 |
| TdT; 30 mins | Too many to count | 10 |

The TdT versus Topo cloning was repeated using the test insert included with the Topo TA cloning kit (Invitrogen, #45-0071).

### Topo Cloning

1 μL diluted salt solution
3 μL gel purified TOPO control PCR
1 μL mQ water
1 μL vector DNA
5 minutes at room temperature, then on ice

### TdT Cloning

0.5 μL 10× TdT buffer (NEB)
2 μL 2.5 mM CoCl2
1 μL 10 mM GTP, 1 mM ATP solution
1 μL pAthena vector (80 ng/μL)
5 μL PCR insert (600 bp; 20 ng/μL)
0.25 μL TdT (NEB)
0.25 μL T4 DNA ligase (NEB)
25° C. for various time points, heat killed at 65° C. for 5 mins

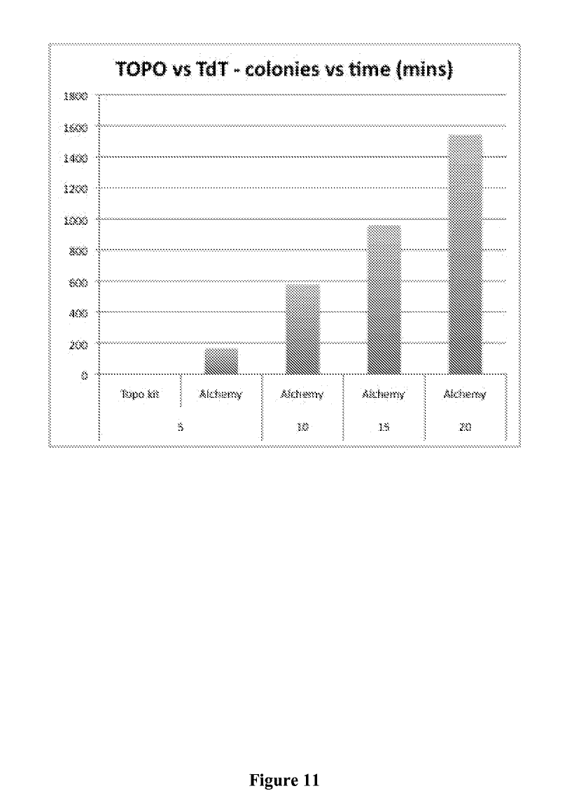

Both ligations were then transformed into Argentum cells and plated, as described above. Table 5 shows the number of colony forming units obtained from each reaction, and the inserts per 10 colonies. The number of colony forming units versus time for each reaction is shown in FIG. 11.

TABLE 5
Comparison of TOPO TA and TdT cloning systems

| Cloning system | Cfu/plate | Inserts/10 colonines |
|---|---|---|
| TOPO; 5 mins | 4 | 3 (of 4) |
| TdT; 5 mins | 234 | 5 |
| 10 mins | 720 | 8 |
| 15 mins | 1069 | 9 |
| 20 mins | 1,540 | 10 |

### Example 10

### Maintenance of Sequence Fidelity of DNA Insert and Vector

Other cloning methods that use non-natural bases have resulted in mutagenic areas around the ligation joint, presumably due to error-prone correction. For example, in Gal et al. (1999), the directional cloning of native PCR products with preformed sticky ends (Autosticky PCR) is described. The authors used abasic sites in the oligonucleotide primers to yield single-stranded tail regions that could be annealed and ligated to complementary vector ends. However, at one end of their sequence the abasic site was deleted entirely 7 of 11 times, and repaired as a dAMP residue 3 of 11 times. Another clone was also mutated.

Two methods were used to ensure that the repair of end sequences by the TdT cloning method of the invention maintained sequence fidelity.

### 1. EcoRV Fragment Cloning

Oligonucleotide synthesis often produces products that have sequence errors as the synthesis process is error prone. Therefore, to demonstrate that the TdT cloning was not producing errors, a product that was 100% sequence perfect at the ends was used.

Using the EcoRV restriction enzyme, which produces blunt products (GATAATC), a PCR product was cloned that had EcoRV ends on both ends. The presence of the EcoRV sites was confirmed by digest. The excised DNA fragment was dephosphorylated with Calf Intestinal Phosphatase (CIP) to produce a product that was functionally equivalent to a PCR product (i.e. 5′ hydroxyl end). This product was cloned by the method of the invention using the Klenow exo-enzyme to add dATPs to the 3′ end, before using TdT/T4 ligase to clone the product into the vector.

Eight clones were sequenced at both ends. 16/16 junctions had perfect fidelity of both the vector ends (CCT) and the EcoRV half-site (ATC). 7 of 16 of the clones were found to have an extra A base in the sequence, which was most likely a result of the residual dATP bases in the TdT reaction being used to extend the 3′ overhang base by TdT before the rGrG dinucleotide addition allowed T4 ligase cloning (i.e. they had a junction of CCT T ATC instead of CCT ATC).

### 2. Bsu36I Half-Site Cloning

Shorter oligonucleotides have fewer base errors than longer oligonucleotides, and therefore the error frequency of cloning was determined by choosing the Bsu36I restriction endonuclease that has a recognition sequence of CCTNAGG.

The CCT sequence may be contributed by the vector end, and the NAGG was contributed by the ends of the PCR primers that had the sequence GAGG. The PCR product was generated by Taq DNA polymerase to give a 3′ dA overhang.

Significantly, it was found that Bsu36I correctly cleaved the insert cloning region in 36 of 40 junctions. The 4 sites (10%) that were not correctly cut are within the error frequency encountered with primer mutations at the ends of PCR products that are directly cloned. This was unexpected because there are 3 non-standard bases in the region of the cloning joint. There are 2 ribobases (rGrG) that are the continuous strand, a non-extendable dideoxy-TMP base opposite the ribobases, as well as a base that lacks a 5′ phosphate. It was expected that these 3 structures might enhance the mutation frequency in the region of the cloning joint as the cell may a) fail to recognise one of the non-standard bases for correction, b) correct the substrates in a repair sequence that results in strand breakage and loss of identity through non-homologous end repair or, c) utilise a DNA repair system that is error prone e.g. SOS repair.

### Example 11

### pAqcuire3 Vector Comprising 3′ Overhangs with 3′ Phosphate

A new vector that has a nicking site at both ends (Nt.BspQI; GCTCTTCNA) was constructed to enable oligonucleotide exchange with oligonucleotides having a 3′ phosphate.

To produce pAcquire3, a vector comprising a cloning region having the sequence as provided in SEQ ID NO:7 was first nicked with Nt.BspQI and then digested with BsmI. Following cleavage by both enzymes (Nt.BspQI and BsmI), the reaction was heated to 80° C. in the presence of a large excess of oligonucleotides that have a 3′ phosphate and that are complementary to the single stranded regions of the vector. The oligonucleotides are:

(SEQ ID NO: 8)
cctcgagcgattacagagaatgcct-3′ phosphate
(SEQ ID NO: 9)
cgaagcttgtatgagtctgaatgcct-3′ phosphate

Once the vector was prepared, it was tested in the following reactions:
1 μl 5× NEB Terminal Transferase Buffer
1 μl 15 mM CoCl₂
1 μl 10 mM GTP:1 mM ATP
1 μl pAcquire3 (100 ng/μl)
5.5 μl PCR insert (alpha β-galactosidase expression cassette; 30 ng/μl)
0.5 μl TdT:ligase blend

The reaction was incubated at 25° C. for 20 minutes, then at 65° C. for 5 minutes. 1 μl of this reaction mix was used to electroporate competent cells. The complete reaction and partial reactions were assembled as described in Table 6:

TABLE 6
TdT cloning using 3′ overhangs with 3′ phosphate

| Reaction | Conditions | #blue:white colonies |
|---|---|---|
| 1) | Vector + enzyme | 0:104 |
| 2) | Vector + insert | 1:315 |
| 3) | Vector + insert + enzyme | 746:164 |
| 4) | Insert + enzyme | 0:0 |
| 5) | Vector + insert + enzyme + | 1702:132 |
| | quench oligonucleotide | |

Due to the cloned PCR product expressing the alpha fragment of β-galactosidase, enabling complementation of the lacZΔM15 host allele, blue colonies were scored as having cloned insert, whites were empty vector. For reaction (5), additional oligonucleotides, complementary to the 3′ phosphate oligonucleotides above, were added as 1 μL of a 50 μM stock to the reaction before heat inactivation.

It was observed that a 3′ phosphate both protects the vector from further TdT modification during the reaction, while maintaining cloning competency.

Furthermore, it was observed that a ligated insert flanked by single stranded vector regions, produced by heating the reaction in an excess of free complementary oligonucleotides SEQ ID NO:8 and 9, had a higher cloning efficiency than inserts flanked by nicked regions.

All publications discussed and/or referenced herein are incorporated herein in their entirety.

The present application claims priority from AU 2008905669 filed 3 Nov. 2008, and AU 2009903350 filed 17 Jul. 2009, the entire contents of both of which are incorporated herein by reference.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Clark (1988) Nucleic Acids Res, 16:9677-96869.
Datsenko and Wanner (2000) Proc Natl Acad Sci USA, 97:6640-6645.
Gal et al. (1999) Mol Gen Genet, 260:569-573.
Holton and Graham (1991) Nucleic Acids Res, 19:1156.
Hu (1993) DNA Cell Bio, 12:763-770.

1. A kit for cloning a dsDNA, the kit comprising nucleotides, terminal transferase and DNA ligase.

2. The kit of claim 1, wherein the nucleotides are ribonucleotides.

3. The kit of claim 1, wherein the kit further comprises a vector comprising at least one 3′ overhang.

4. The kit of claim 3, wherein the end of the at least one 3′ overhang of the vector comprises a chain-terminating base analogue or a 3′ phosphate.

5. (canceled)

6. The kit of claim 3, wherein the vector comprises at least one 3′ overhang of one to four bases.

7-8. (canceled)

9. The kit of claim 1, wherein the DNA ligase is T4 DNA ligase.

10-12. (canceled)

13. A composition comprising terminal transferase and DNA ligase.

14. (canceled)

15. The composition of claim 13, wherein the DNA ligase is T4 DNA ligase.

16. (canceled)

17. A method of cloning a dsDNA molecule, the method comprising: i) producing a dsDNA molecule comprising 3′ overhangs by contacting a dsDNA molecule with terminal transferase in the presence of ribonucleotides, ii) ligating the dsDNA molecule comprising 3′ overhangs to a vector comprising at least one complementary 3′ overhang, and iii) transforming the dsDNA molecule ligated to the vector into a host cell.

18. The method of claim 17, wherein the dsDNA molecule comprises a single base 3′ overhang prior to contacting the dsDNA molecule with terminal transferase in the presence of ribonucleotides.

19-20. (canceled)

21. The method of claim 17, wherein one to four ribonucleotides are added to the 3′ ends of the dsDNA molecule.

22. The method of claim 21, wherein two ribonucleotides are added to the 3′ ends of the dsDNA molecule.

23. The method of claim 17, wherein contacting the dsDNA molecule with terminal transferase in the presence of ribonucleotides and ligating the dsDNA molecule to the vector are performed in the same buffer.

24. (canceled)

25. The method of claim 23, wherein contacting the dsDNA molecule with terminal transferase in the presence of ribonucleotides and ligating the dsDNA molecule to the vector are performed concurrently in the same reaction mixture, wherein the at least one 3′ overhang of the vector is non-reactive with terminal transferase.

26. A method of cloning a dsDNA molecule, the method comprising:
i) producing a dsDNA molecule comprising 3′ overhangs by contacting a dsDNA molecule with terminal transferase in the presence of nucleotides,
ii) ligating the dsDNA molecule comprising 3′ overhangs to a vector comprising at least one complementary 3′ overhang, and
iii) transforming the dsDNA molecule ligated to the vector into a host cell,
wherein contacting the dsDNA molecule with terminal transferase in the presence of nucleotides and ligating the dsDNA molecule to the vector are performed concurrently in the same reaction mixture, and wherein the at least one 3′ overhang of the vector is non-reactive with terminal transferase.

27. The method of claim 26, wherein the nucleotides are ribonucleotides.

28. The method of claim 26, wherein the ends of the at least one 3′ overhang of the vector comprises a chain-terminating base analogue or 3′ phosphate.

29. The method of claim 28, wherein the chain-terminating base analogue is a dideoxynucleotide or an acyclonucleotide.

30-32. (canceled)

33. The method of claim 17, wherein the vector is a plasmid.

34. The method of claim 17, wherein the host cell is E. coli.

35. (canceled)
